(19) United States
(12) Patent Application Publication
(10) Pub. No.: US 2026/0033848 A1
(43) Pub. Date: Feb. 5, 2026

(54) INTRAVASCULAR DUAL FREQUENCY SONOTHROMBOLYSIS MEDIATED WITH MICROBUBBLES/NANODROPLETS

(71) Applicants: North Carolina State University, Raleigh, NC (US); The Regents of the University of Michigan, Ann Arbor, MI (US); The University of North Carolina at Chapel Hill, Chapel Hill, NC (US)

(72) Inventors: Xiaoning Jiang, Cary, NC (US); Zhen Xu, Ann Arbor, MI (US); Bohua Zhang, Raleigh, NC (US); Huaiyu Wu, Raleigh, NC (US); Jinwook Kim, Chapel Hill, NC (US); Howuk Kim, San Jose, CA (US); Paul Alexander Dayton, Carrboro, NC (US); Leela Goel, Raieigh, NC (US)

(21) Appl. No.: 19/100,114

(22) PCT Filed: Aug. 7, 2023
(86) PCT No.: PCT/US2023/029628

§ 371 Date: Jan. 30, 2025

Related U.S. Application Data
(60) Provisional application No. 63/395,835, filed on Aug. 7, 2022.

(51) Int. Cl. A61B 17/22

(52) U.S. Cl. CPC A61B 17/2202; A61B 2017/22084; A61B 2017/22088; A61B 2017/22089

(57) ABSTRACT

A method for sonothrombolysis mediated with contrast agents includes administering at least one contrast agent into a blood vessel of a patient. The method further includes controlling application of ultrasound energy to the at least one contrast agent within the blood vessel, wherein controlling the application of the ultrasound energy includes driving an ultrasound transducer with a signal having a first frequency component and a second frequency component different from the first frequency component.

### CROSS REFERENCE TO RELATED APPLICATIONS

This application claims the benefit of and priority to U.S. Provisional Patent Application Ser. No. 63/395,835 entitled “INTRAVASCULAR DUAL FREQUENCY SONOTHROMBOLYSIS MEDIATED WITH MICROBUBBLES/NANODROPLETS,” filed Aug. 7, 2022, the disclosure of which is incorporated herein by reference in its entirety.

### GOVERNMENT INTEREST

This invention was made with government support under grant numbers EB027304 and HL141967 awarded by the National Institutes of Health. The government has certain rights in the invention.

### TECHNICAL FIELD

The subject matter described herein relates to sonothrombolysis. More particularly, the subject matter described herein relates to intravascular dual frequency sonothrombolysis mediated with microbubbles and/or nanodroplets

### BACKGROUND

Venous thromboembolism (VTE) is one of the leading causes of death worldwide. Deep vein thrombosis (DVT) may cause pulmonary embolism (PE) when the clot debris freely enters the arteries of the lungs, which often results in sudden death. VTE affects 300,000 to 600,000 patients every year and one-third of the DVT results in PE with more morbidity and mortality. One of the most common treatments for blood clots is systemic thrombolysis using a tissue plasminogen activator (tPA). However, the treatment usually takes over 24 hours to be effective, which results in a high risk of intracranial bleeding. Other traditional treatment methods, including mechanical thrombectomy, suffer from the risks of blood vessel damage and pulmonary embolism with large clot debris.

Recent studies have reported ultrasound-enhanced thrombolysis, or sonothrombolysis, as the alternative to the traditional treatment methods with improved thrombolysis rate and safety. With contrast agents such as microbubbles (MBs), sonothrombolysis has been reported to be effective for unretracted clots. The primary mechanism is that MBs can generate stable and inertial cavitation under ultrasound excitation, which induces microstreaming and microjets for breaking the structure of the blood clots. However, microbubble-mediated sonothrombolysis has very limited effectiveness in treating retracted clots.

Sonothrombolysis can be delivered using external ultrasound, although this approach is significantly affected by the overlying tissue path, which may induce significant attenuation or aberration of ultrasound propagation. Moreover, other concerns may include skin burns or vessel damage either due to poor acoustic coupling at the skin-transducer interface or the respiratory movement from organs with high electric power. Sonothrombolysis can also be delivered with an intravascular transducer. Intravascular sonothrombolysis is challenging, because a sufficiently high pressure needs to be achieved to generate cavitation, while the ultrasound power output is proportional to the surface area of the ultrasound transducer. The intravascular transducer has a very small surface area, thus is capable of generating lower pressure. Prior studies have shown side-viewing intravascular transducer with a single frequency, which is only effective for a partially occluded clot with a small opening in the center for the transducer to be inserted through.

### SUMMARY

Methods and systems for sonothrombolysis mediated with contrast agents are disclosed. A method for sonothrombolysis mediated with contrast agents includes administering at least one contrast agent into a blood vessel of a patient. The method further includes controlling application of ultrasound energy to the at least one contrast agent within the blood vessel, wherein controlling the application of the ultrasound energy includes driving an ultrasound transducer with a signal having a first frequency component and a second frequency component different from the first frequency component.
In an aspect of the disclosed method, the method includes inserting the ultrasound transducer into the blood vessel adjacent to the at least one contrast agent before controlling the application of the ultrasound energy.
In an aspect of the disclosed method, the ultrasound transducer is housed in a catheter.
In an aspect of the disclosed method, the first frequency component is a constant frequency component and the second frequency component includes a plurality of frequency steps within a range of frequencies.
In an aspect of the disclosed method, the first frequency component has a center frequency of about 750 kHz and the frequency steps range from about 450 kHz to about 650 kHz at 50 kHz intervals.
In an aspect of the disclosed method, the at least one contrast agent comprises at least one nanodroplet and/or at least one microbubble.
In an aspect of the disclosed method, a strength of the signal is adjusted according to each frequency component.
In an aspect of the disclosed method, the strength of the signal is adjusted to provide approximately equal levels of ultrasound energy by the first frequency component and the second frequency component of the signal.
In an aspect of the disclosed method, the ultrasound transducer comprises at least one piezoelectric element.
In an aspect of the disclosed method, the ultrasound transducer comprises a forward-viewing ultrasound transducer.
A system for sonothrombolysis mediated with contrast agents includes an ultrasound transducer. The system further includes a function generator connected to the ultrasound transducer, the function generator configured for controlling application of ultrasound energy to at least one contrast agent within a blood vessel, wherein controlling the application of the ultrasound energy includes driving the ultrasound transducer with a signal having a first frequency component and a second frequency component different from the first frequency component.
In an aspect of the disclosed system, the ultrasound transducer is configured for being inserted into the blood vessel adjacent to the at least one contrast agent.
In an aspect of the disclosed system, the ultrasound transducer is housed in a catheter.
In an aspect of the disclosed system, the first frequency component is a constant frequency component and the second frequency component includes a plurality of frequency steps within a range of frequencies.
In an aspect of the disclosed system, the first frequency component has a center frequency of about 750 KHz and the frequency steps range from about 450 kHz to about 650 kHz at 50 KHz intervals.
In an aspect of the disclosed system, the at least one contrast agent comprises at least one nanodroplet and/or at least one microbubble.
In an aspect of the disclosed system, the function generator is configured for adjusting a strength of the signal according to each frequency component.
In an aspect of the disclosed system, the function generator is configured for adjusting the strength of the signal to provide approximately equal levels of ultrasound energy by the first frequency component and the second frequency component of the signal.
In an aspect of the disclosed system, the ultrasound transducer comprises at least one piezoelectric element.
In an aspect of the disclosed system, the ultrasound transducer comprises a forward-viewing ultrasound transducer.

As used herein, the term “unretracted” blood clot refers to a blood clot that is not retracted.

### BRIEF DESCRIPTION OF THE DRAWINGS

FIG. 1 shows an example system for sonothrombolysis mediated with contrast agents;

FIG. 2A shows active layers of an ultrasound transducer being stacked and bonded;

FIG. 2B shows a matching layer being applied to active layers of an ultrasound transducer;

FIG. 2C shows active layers with a matching layer being diced;

FIG. 2D shows an isolation layer applied to the sides of an ultrasound transducer;

FIG. 2E shows electrodes connected to an ultrasound transducer;

FIG. 2F shows wires attached and a passivation layer applied to an ultrasound transducer;

FIG. 3A shows an example setup for a pulse-echo test;

FIG. 3B shows an example setup for a pressure output test;

FIG. 4 shows an example setup of a flow model for an in vitro microbubbles mediated intravascular sonothrombolysis tests;

FIG. 5 shows a chart of design parameters for each layer of an example ultrasound transducer;

FIG. 6A shows a graph representing structures of an ultrasound transducer with symmetric simulation methods;

FIG. 6B shows a graph of sound pressure with 80-V input;

FIG. 7A shows a graph of impendence and phase angle for an ultrasound transducer;

FIG. 7B shows a graph of measured pulse-echo results for an ultrasound transducer;

FIG. 8A shows a graph of the measured PTP and PNP from the chosen single frequency of 750 kHz under an input voltage of 80 Vₚₚ;

FIG. 8B shows a graph of the measured pressure level from various frequency combinations with a ratio of 1:1 to each frequency component;

FIG. 9A shows a graph of mass reduction for unretracted and retracted clots under frequency combinations;

FIG. 9B shows a graph of dual-frequency treatment improvement compared with single-frequency treatment;

FIG. 10A shows a graph of passive cavitation dose under various frequency combinations;

FIG. 10B shows a graph of cavitation dose improvement with dual-frequency excitation; and

FIG. 11 is a flow diagram illustrating example method for sonothrombolysis mediated with contrast agents.

### DETAILED DESCRIPTION

The subject matter described herein includes methods and systems for sonothrombolysis mediated with contrast agents. An ultrasound transducer is configured to perform intravascular ultrasound-enhanced thrombolysis, or sonothrombolysis, to treat DVT using dual-frequency ultrasound excitation. The ultrasound transducer can be integrated into a catheter with a lumen to deliver contrast agents directly to the surface of a blood clot for more effective clot lysis. Notably, the dual-frequency sonothrombolysis treatment with contrast agents (CAs) is more efficient than single-frequency treatment in trigging the acoustic cavitation by decreasing the cavitation threshold, which leads to a higher clot lysis rate. The ultrasound transducer can be forward-viewing. The surface area available for a forward-viewing intravascular transducer is smaller than the side-viewing intravascular transducer. Existing devices either have problems in covering a large enough frequency range or providing enough pressure output with limited aperture size, which are required for dual-frequency sonothrombolysis with one intravascular device.

Deep vein thrombosis typically is treated with a tissue plasminogen activator (IPA) or mechanical thrombectomy, which may suffer from vessel damage or hemorrhage. Ultrasound-enhanced thrombolysis with external transducers required high pressure or power and may suffer from skin burn or tissue damage. The disclosed ultrasound transducer decreases the threshold of the contrast agents and provides more effective treatment over the retracted clot. The changes over the excitation method can be easily programmed in advance and controlled by the operators. The forward-viewing intravascular ultrasound transducer can treat completely occluded clots, compared to the side-viewing ultrasound transducer only capable of treating partially occluded clots. The ultrasound transducer enables the enhanced capability of safe, simple, and efficient deep venous thrombolysis for therapies in hospitals.

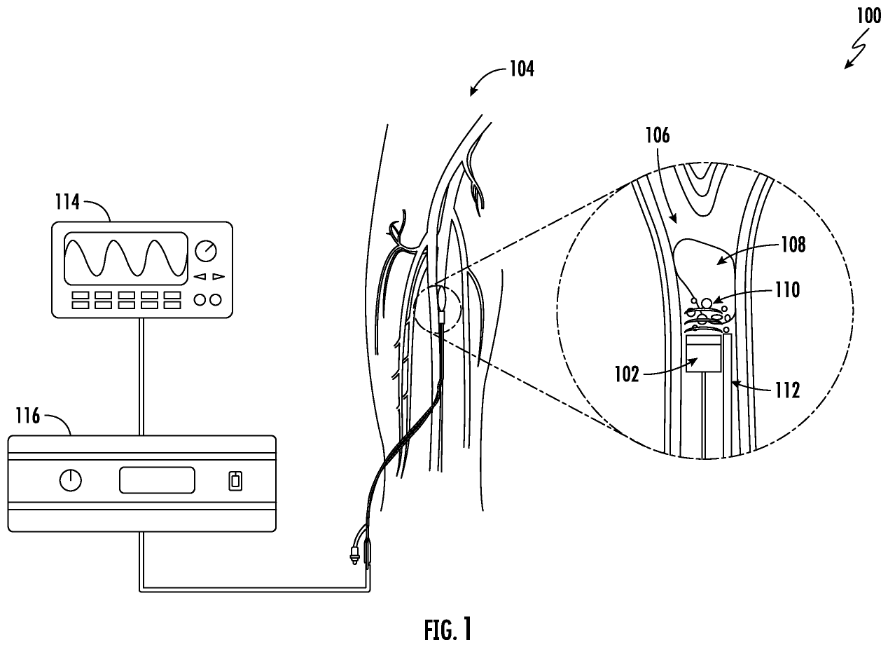

FIG. 1 shows an example system 100 for sonothrombolysis mediated with contrast agents. System 100 can be made portable to allow high-risk patients for clot treatment in their own homes. An ultrasound transducer 102 may be positioned within a blood vessel 106 of a patient 104 to emit ultrasound energy targeting a blood clot 108 in the blood vessel 106. Blood clot 108 may be positioned in a deep vein, i.e., DVT. Ultrasound transducer 102 can be used with contrast agents 110 and positioned adjacent to a surface of blood clot 108. Ultrasound transducer 102 can be forward-viewing, side-viewing, or a combination of forward-viewing and side-viewing. An example advantage of a forward-viewing transducer is that it can better target unretracted clots than side-viewing transducers. Ultrasound transducer 102 can be inserted into blood vessel 106 with a contrast agents injection tube 112 configured for emitting contrast agents 110. Contrast agents 110 can include microbubbles (MBs) and/or nanodroplets (NDs). Ultrasound transducer 102 can be inserted into blood vessel 106 adjacent to contrast agents 110. Contrast agents injection tube 112 can deliver contrast agents 110 directly to a surface of blood clot 108. Contrast agents injection tube 112 can deliver contrast agents 110 at a surface of blood clot 108 closest to ultrasound transducer 102 such that the contrast agents 110 are positioned between the ultrasound transducer 102 and the blood clot 108.

In an aspect of the disclosed subject matter, ultrasound transducer 102 can be housed in a catheter. The catheter may include a lumen through which ultrasound transducer 102 emits ultrasound energy. The catheter may also house contrast agents injection tube 112 and include a lumen, either the same lumen discussed or a second separate lumen, through which the contrast agents injection tube 112 can emit contrast agents 110. Ultrasound transducer 102 can be used with a miniaturized imaging transducer or array to help monitor and image the clot dissolution efficiency in real-time. Ultrasound transducer 102 can be forward-viewing or combined forward-viewing and side-viewing. Ultrasound transducer 102 can be integrated into the catheter with or without a guide wire.

Ultrasound transducer 102 can be connected to a function generator 114 configured to transmit a signal to the ultrasound transducer 102 that causes the ultrasound transducer 102 to generate ultrasound energy, i.e., acoustic energy. Function generator 114 controls application of ultrasound energy to at least one contrast agent 110 within blood vessel 106. Function generator 114 controls the application of the ultrasound energy by driving ultrasound transducer 102 with a signal. The type of signal from function generator 114 can be compatible with the type of ultrasound transducer 102 implemented. For example and without limitation, the signal transmitted from function generator 114 can include a light signal when ultrasound transducer 102 includes an optical ultrasound transducer, an electrical signal when ultrasound transducer 102 includes a piezoelectric ultrasound transducer, an acoustic signal when ultrasound transducer 102 includes an acoustic ultrasound transducers, and the like.

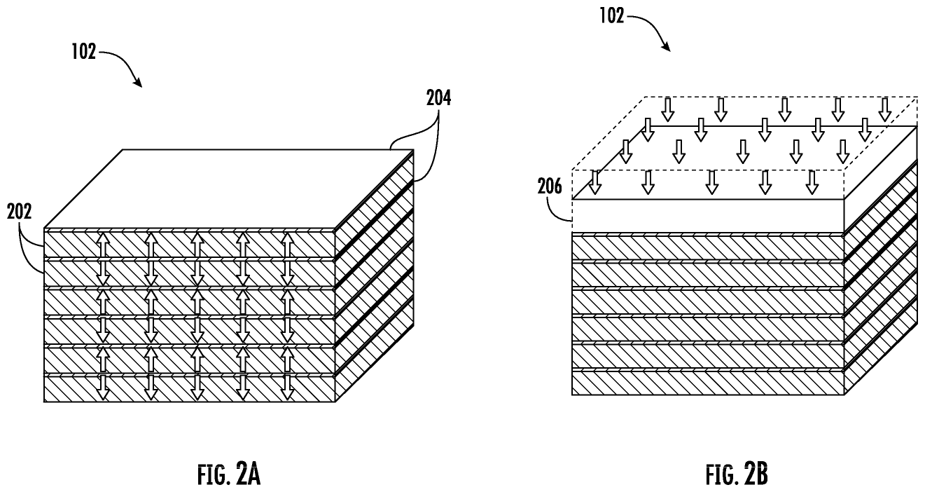

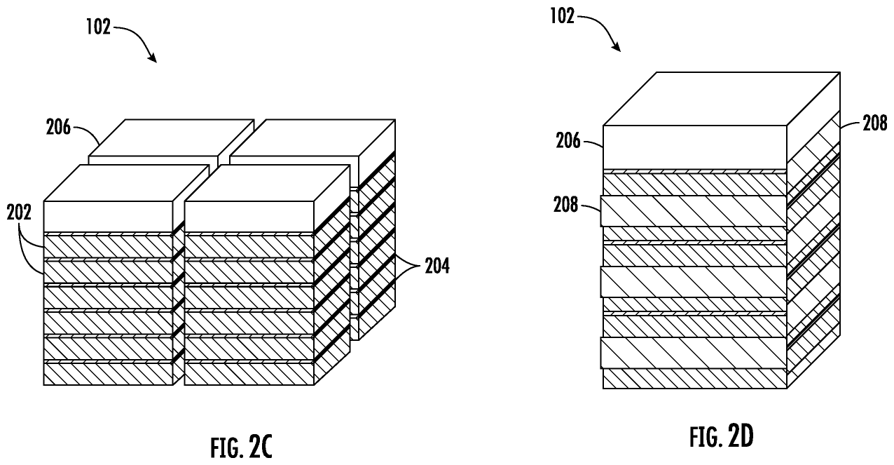

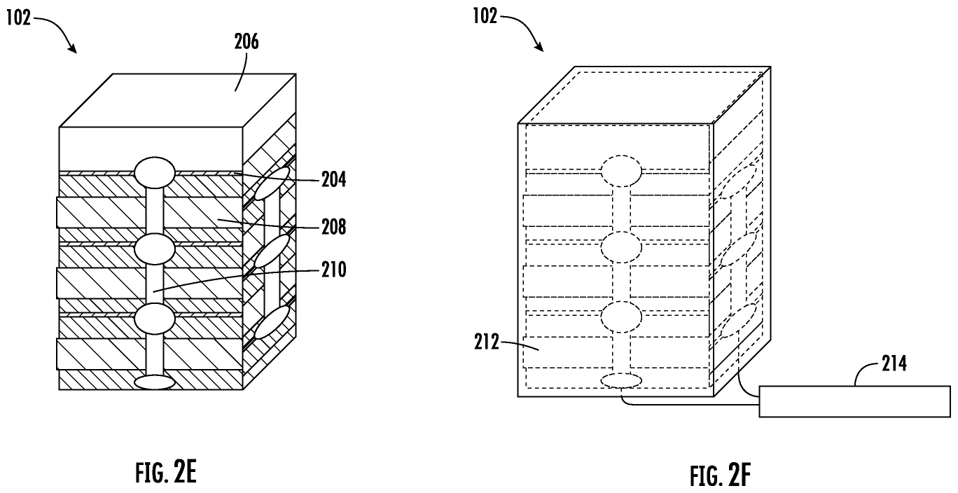

The signal transmitted from function generator 114 has a first frequency component and a second frequency component different from the first frequency component. The first frequency component and the second frequency component cause ultrasound transducer 102 to generate ultrasound energy with corresponding first frequency component and second frequency component. The frequencies of the ultrasound energy can be fine-tuned as desired. Function generator 114 can be controlled to adjust the first frequency component and the second frequency component of the signal. The first frequency component can be the center frequency of ultrasound transducer 102, which provides an efficient conversion from the signal strength to ultrasound energy. A center frequency of ultrasound transducer 102 is based on the dimensions of the ultrasound transducer 102 which can be determined altered to produce the intended center frequency, such as lapping or shaping a matching layer 206 shown in FIG. 2B on the ultrasound transducer 102 to obtain a desired overall length of the ultrasound transducer 102. The first and second frequency components can include frequencies up to about 10 MHz. In an aspect of the disclosed subject matter, the first and second frequency components of the signal can range from about 400 KHz to about 10 MHz. As an example, and without limitation, the first and second frequency components of the signal can each be one of 450 kHz, 500 kHz, 550 kHz, 600 kHz, 650 kHz, 700 KHz, 750 KHz, or 800 KHz. In one aspect of ultrasound transducer 102, the center frequency is about 750 kHz, which is the selected first frequency component of the signal, and the second frequency component can be selected from a range of about 450 kHz to about 650 KHz at 50 kHz intervals. The signal generated by function generator 114 can be generated as a pulse signal, wherein an operator can control a duty cycle and/or a pulse repetition frequency (PRF) of the signal. For example and without limitation, the pulse may include a duty cycle of about 5% and a PRF of 200 Hz.

System 100 may include a power amplifier 116 that receives the signal from function generator 114, amplifies the signal, and transmits the amplified signal to ultrasound transducer 102 with a driving voltage to produce ultrasound energy. Power amplifier 116 may strengthen a received signal with high enough voltage and power output to cause a desired level of ultrasound energy and pressure from ultrasound transducer 102. The driving voltage may be controlled by function generator 114 and/or power amplified 116 and may be, for example, about 70 V, 75 V, 80 V, 85 V, or 90 V peak-to-peak. In one aspect, the driving voltage may be constant. In another aspect, the driving voltage may fluctuate based on the frequency component. Ultrasound transducer 102 generates different pressure outputs under the same driving voltage of the received signal according to the frequency component. Function generator 114 and/or power amplifier 116 may control a strength of the signal transmitted to ultrasound transducer 102. The strength of each frequency component of the signal can be adjusted to produce approximately equal levels of ultrasound energy from each frequency component.

Using dual-frequency insonation generated by ultrasound transducer 102 within the FDA limits and contrast agents 110 to enhance the treatment, physicians can adjust the ultrasound exposure to blood clot 108 as necessary. Ultrasound transducer 102 can achieve lower excitation peak negative pressure (PNP) for MB-mediated sonothrombolysis intravascularly compared to external transducers, while avoiding vessel wall damage. Dual-frequency excitation facilitates significant cavitation improvement even with relatively low pressure (PNP<1.5 MPa) generated by an intravascular transducer, such as ultrasound transducer 102, and consequently enhances sonothrombolysis efficacy. As such, various frequency combinations with ultrasound transducer 102 can be adjusted in terms of cavitation dose and thrombolytic efficiency for both unretracted and retracted thrombosis treatment in a deep vein. By using a high-frequency and low-frequency combination, improved cavitation energy can be achieved by lower input energy compared to using low-frequency excitation alone.

FIGS. 2A-2F show the design and fabrication of an example aspect of ultrasound transducer 102, which includes a piezoelectric transducer with stacked piezoelectric elements. Although the present disclosure describes embodiments where ultrasound transducer 102 includes piezoelectric ultrasound transducers, the ultrasound transducer 102 is not limited to this type of transducer. Ultrasound transducer 102 disclosed may include, for example, piezoelectric ultrasound transducers, optical ultrasound transducers, acoustic ultrasound transducers, or any other ultrasound transducers known in the art consistent with this disclosure.

FIG. 2A shows active layers 202 of ultrasound transducer 102 being stacked and bonded. Active layers 202 may include, for example, layers of piezoelectric elements. In this example, active layers 202 each include a PZT-5A transducer with a thickness of about 250 μm and stacked together. Considering the small aperture size of ultrasound transducer 102, a multi-layer design can provide enough pressure output with a relatively low electrical impedance. In the example illustrated, active layers 202 includes six stacked layers. In other aspects of the disclosed subject matter, ultrasound transducer 102 may include 2, 3, 4, 5, 7, or more stacked active layers 202. Stacking active layers 202 increases the capacitance and lowers electrical impedance for better electrical impedance matching with the driving electronics, which consequently increased the pressure output under the same excitation conditions. The ultrasound pressure generated from the stacked active layers 202 can equal the summation of the pressure generated by each layer in the active layers 202. Active layers 202 may include piezoelectric elements PZT5A (Type III 301, TRS Technologies, Inc., State College, PA, USA) with relatively high piezoelectric coefficients. Between active layers 202 may be applied bonding layers 204. Bonding layer 204 may include any electrically conductive adhesive, for example a conductive metallic epoxy such as a silver epoxy (E-Solder 3022, Von-Roll Inc., Cleveland, OH, USA). A continuous pressure may be applied to active layer 202 to control a thickness of bonding layer 204. In an aspect of the disclosed subject matter, the thickness of bonding layer 204 may be controlled to about 25 μm.

FIG. 2B shows a matching layer 206 being applied to active layers 202 of ultrasound transducer 102. Matching layer 206 may include for example Al₂O₃/epoxy. Matching layer 206 is lapped or otherwise shaped to a specific thickness according to a desired frequency for ultrasound transducer 102 to generate. In the present example, matching layer 206 is lapped to about 700 μm for the designed center frequency of 750 KHz for ultrasound transducer 102.

FIG. 2C shows active layers 202 with matching layer 206 being diced according to a desired size. In an example aspect of the disclosed subject matter, ultrasound transducer 102 is diced to have an aperture dimension of about 1.4 mm×1.4 mm which is small enough for intravascular applications, for example 9 French or smaller. In other aspects of the disclosed subject matter, the aperture dimension may be smaller or larger than 1.4 mm×1.4 mm.

FIG. 2D shows an isolation layer 208 applied to the sides of ultrasound transducer 102. Isolation layer 208 may include Al₂O₃/epoxy, which is moldable before curing process with a restively large electrical resistance. Applying Al₂O₃/epoxy to isolation layer 208 can provide a higher breakdown voltage and improve reliability of ultrasound transducer 102.

FIG. 2E shows electrodes 210 connected to ultrasound transducer 102. Electrodes 102 are connected to two sides of ultrasound transducer 102 with an adhesive with a low electric resistance, for example silver epoxy, which has an electric resistance of less than 10.

FIG. 2F shows wires 214 attached to ultrasound transducer 102 to enable an electrical connection. A passivation layer 212 is also applied to ultrasound transducer 102. Passivation layer 212 can cover the entire ultrasound transducer 102 including wire 214. In other aspects of the disclosed subject matter, passivation layer 212 can partly cover ultrasound transducer 102. In this example, passivation layer 212 includes a 13-μm parylene layer. The whole of ultrasound transducer 102 can have a diameter of less than 10 French, which is flexible and easily conforms to existing catheters for intravascular applications.

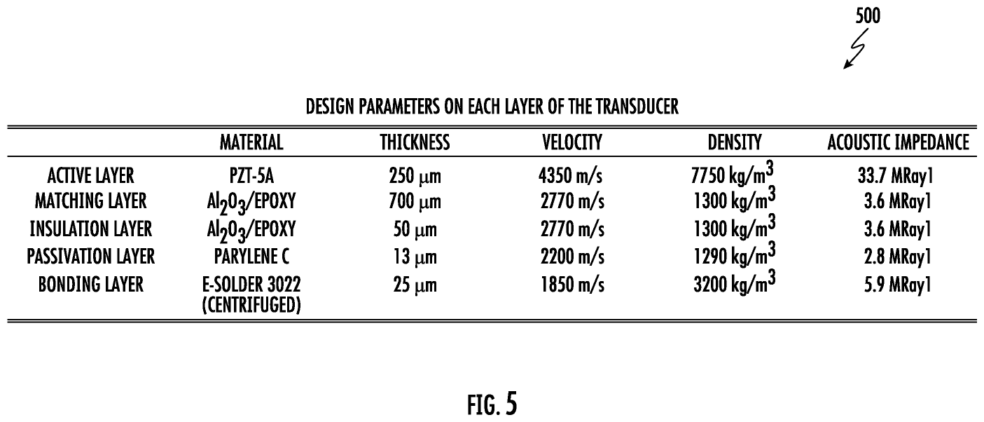

FIG. 5 shows a chart 500 of design parameters for each layer of an example ultrasound transducer, namely the active layer, matching layer, insolation layer (or isolation layer), passivation layer, and bonding layer.

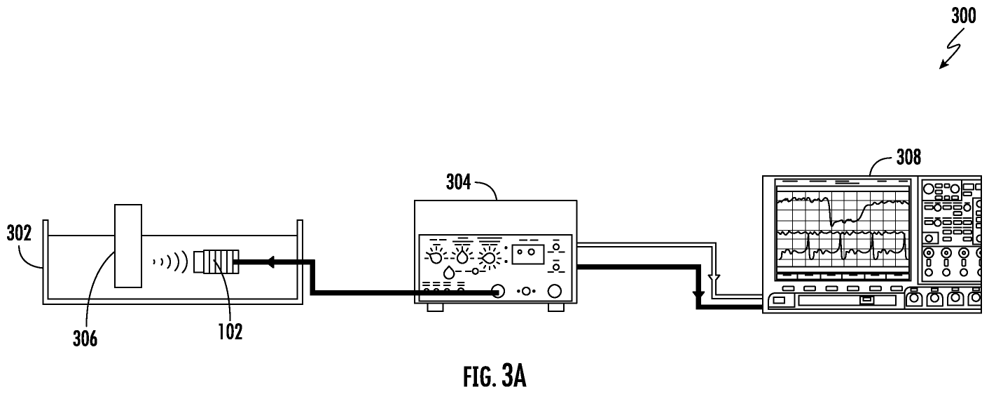

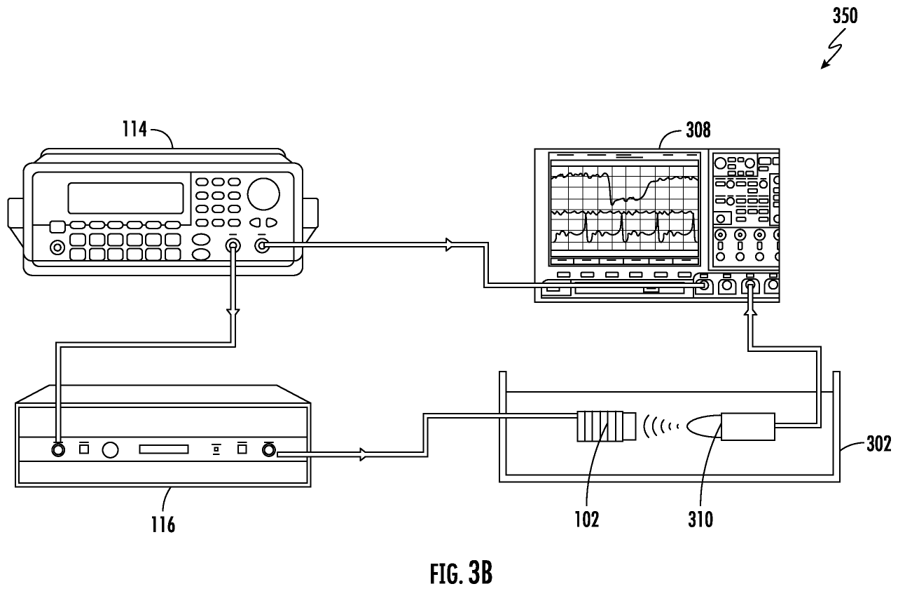

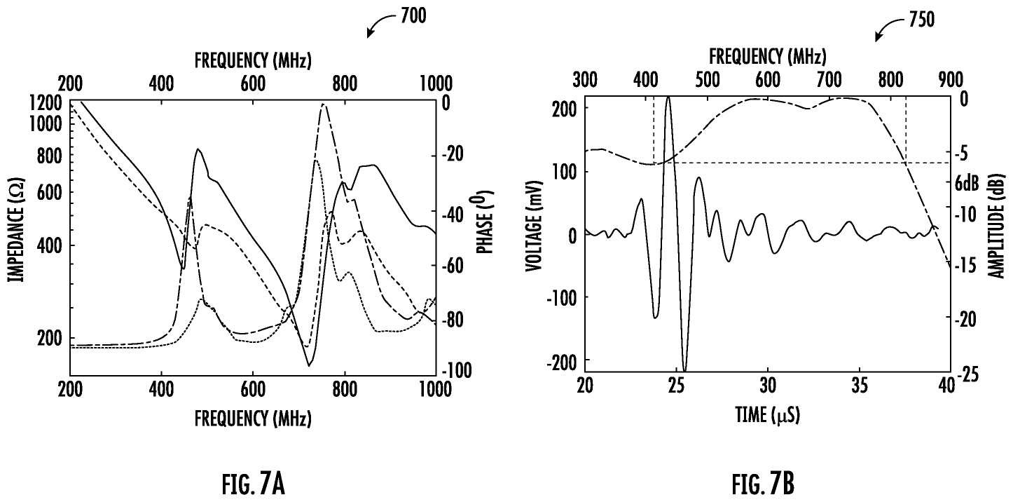

FIG. 3A shows an example setup 300 for a pulse-echo test. To characterize the bandwidth of ultrasound transducer 102 and determine the combinations of each component, the pulse-echo test was first carried out with ultrasound transducer 102 in a water tank 302. A pulser/receiver 304 (5077 PR, Olympus, WA, USA) was used to excite ultrasound transducer 102 with a PRF of 200 Hz and pulse energy of 1 μJ. A steel bar 306 was used as the reflector at a distance of 18 mm. The radio frequency (RF) signal was collected with an oscilloscope 308 (DSO7104B, Agilent Technologies, Santa Clara, CA, USA). The measured pulse-echo signal was used to obtain the bandwidth of ultrasound transducer 102. Results from the pulse-echo test are shown in FIG. 7B.

FIG. 3B shows an example setup 350 for a pressure output test. To measure the pressure output induced by ultrasound transducer 102, function generator 114 (33250A, Agilent Tech. Inc., Santa Clara, CA, USA) was first connected to power amplifier 116 with a power gain of 28 dB (75A250A, AR, Souderton, PA, USA). Then, ultrasound transducer 102 was excited with a sinusoidal pulse of ten cycles per 10 ms in water tank 302. A 20-dB preamplifier (AH-2020, ONDA Corporation, Sunnyvale, CA, USA) and a hydrophone 310 (HGL-0085, ONDA Corporation, Sunnyvale, CA, USA) were used to collect the output signal. The pressure outputs under different frequencies were measured.

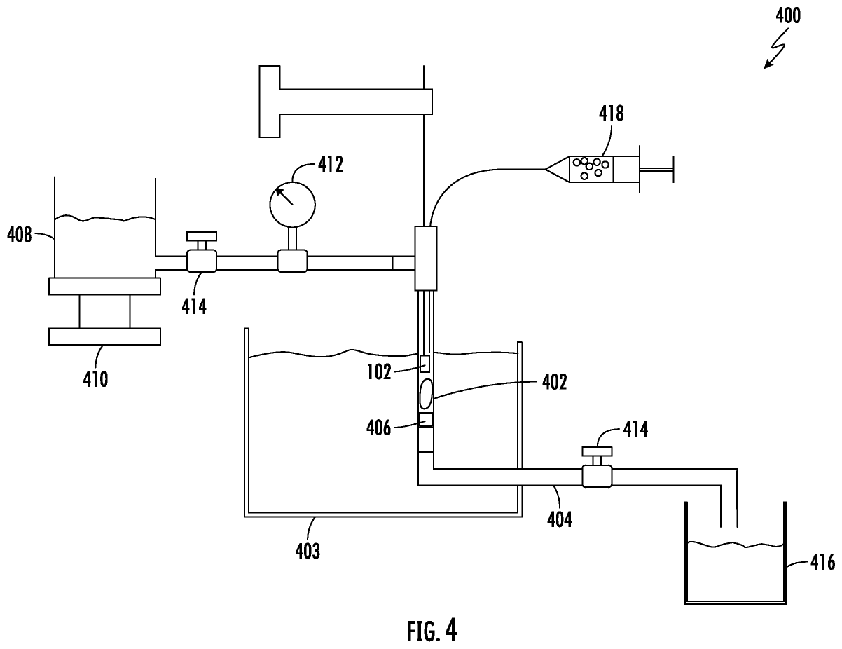

FIG. 4 shows an example setup of a flow model 400 for in vitro microbubbles mediated intravascular sonothrombolysis tests. Retracted and unretracted blood clot samples 402 were first prepared. The acid citrate dextrose (ACD) bovine blood (Densco Marketing, Inc., Woodstock, IL, USA) was added with the 2.75% W/V calcium chloride solution (Fisher Scientific, Fair Lawn, NJ, USA) with a volume ratio of 10:1. For retracted clots, the mixture was drained into borosilicate glass pipettes and sealed. For the unretracted clots, the mixture was transferred to plastic microcentrifuge tubes and sealed. The containers were immersed in a water bath 403 with a temperature of 37° C. for 3 hours. After that, all the samples were stored under 4 C for three days for full incubation. In each test, both retracted and unretracted samples were cut into a length of 15 mm with an average weight of 200±20 mg.

Flow model 400 was constructed to mimic the blood vessel with plastic tubes (ID =5 mm). As shown in FIG. 4, a blood clot sample 402 was placed in a tube 404 with a stopper 406 comprising a mesh-shaped fabric to partially block blood clot sample 402 from flowing away and retain the required hydraulic pressure level (0.5 kPa). A water tank 408 holding water and elevated by a height controller 410 was connected to tube 404 to feed water and apply fluidic pressure within the tube 404. By controlling the height of water tank 408, the pressure was kept the same during the experiment. A pressure gauge 412 was connected to tube 404 downflow of water tank 408 to monitor and/or control the pressure. The flow of the water in tube 404 can further be controlled by including a valve 414 on tube 404 upflow and/or downflow from blood clot sample 402 and an end of the tube can release water as needed into a collector 416. A bubble injection 418 was connected to tube 404 and configured to inject MBs into the tube 404 by blood clot sample 402.

For dual-frequency tests, the driving signal consisted of the center frequency of ultrasound transducer 102 (750 kHz in this example) and the second frequency component, which was ranged from 450 to 650 kHz with the 50-KHz interval (750+450, 750+500, 750+550, 750+600, and 750+650 kHz). For each combination, the MBs were injected as the agents with a concentration of 10⁸/mL at a flow rate of 0.1 mL/min. For each input condition, three samples were used to get the averaged clot lysis rate. For each blood clot sample 402, a 30-min treatment was tested. Function generator 114 (AFG 3000, Tektronix, Beaverton, OR, USA) was first connected to the power amplifier 116 (75A250A, AR, Souderton, PA, USA) (shown in FIG. 1). Then, with the same power input, a pulse signal was generated with a duty cycle of 5% and a PRF of 200 Hz. Before and after the test, the mass of blood clot sample 402 was measured for the lysis rate estimation.

Since ultrasound transducer 102 had various pressure outputs under the same driving voltage at the frequency range of 450 KHz-750 KHz, weighting coefficients were applied to ensure that each frequency component generated the same power based on the pressure output characterization results. The dual-frequency waveform was generated with ArbExpress signal generator software (ArbExpress Tektronix Inc., Beaverton, OR, USA) and then transferred into the function generator with a USB connection as the input. To make the input power consistent for each frequency combination, the power was measured with the ultrasound power meter (MODEL UPM-DT-1AV, Ohmic Instruments, St Charles, MO, USA) and the power was kept for 43.8 mW for a fair comparison.

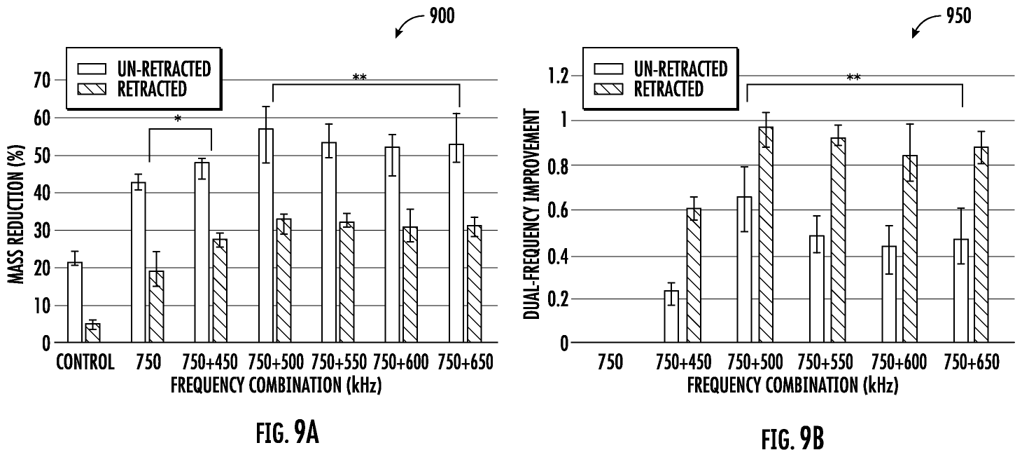

The effectiveness of the ultrasound treatment was defined by the difference in the ratio between the ultrasound treatment and the control groups with Rₑ=(Mᵤ−M_{c})/M_{c} where M_{c} represents the mass reduction in the control group and Mᵤ represents the mass reduction with ultrasound. Then, the ratio difference between the dual-frequency treatment cases and the single-frequency treatment cases was defined as the dual-frequency improvement (improvement=(R_{ed}−Rₑₛ)/Rₑₛ). Res represents the effectiveness ratio with single-frequency treatment, whereas Red represents the effectiveness ratio with dual-frequency treatment. Results from the in vitro MBs mediated intravascular sonothrombolysis tests are shown in FIGS. 9A and 9B.

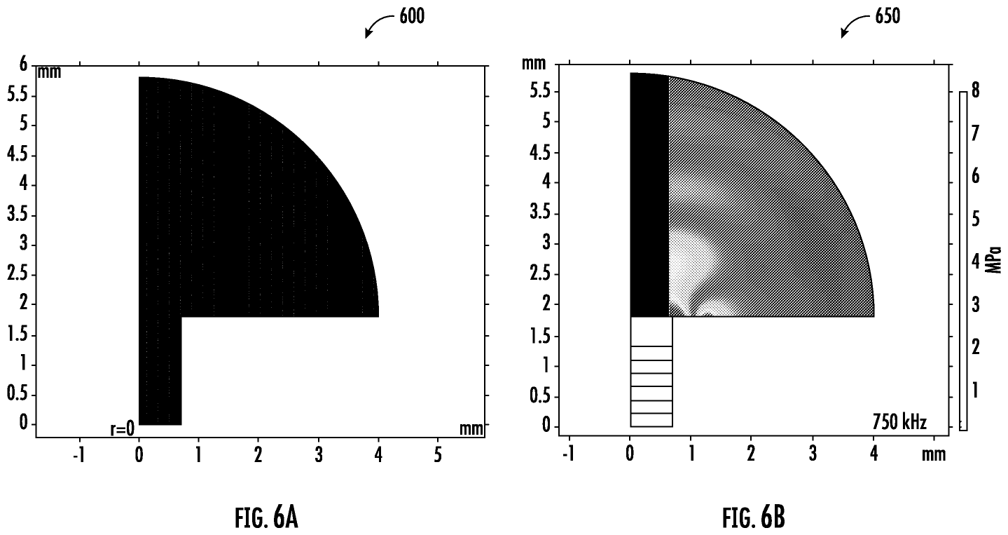

FIGS. 6A and 6B show a simulated sound pressure level with the COMSOL Multiphysics (COMSOL, Burlington, MA, USA). The natural focal depth was estimated to be around 0.8 mm. FIG. 6A shows a graph 600 representing structures of ultrasound transducer 102 with symmetric simulation methods. FIG. 6B shows a graph 650 of sound pressure with 80-V input. As shown in FIG. 6B. the averaged peak-to-peak pressure (PTP) near the focal zone was around 5.3 MPa. The simulation results indicate the capability of ultrasound transducer 102 to reach a high-pressure output.

FIG. 7A shows a graph 700 of impendence and phase angle for ultrasound transducer 102. FIG. 7A shows the designed and measured electrical impedance spectra of ultrasound transducer 102. The results show a consistent trend in the impedance level and the center frequency.

FIG. 7B shows a graph 750 of measured pulse-echo results for ultrasound transducer 102 in setup 300 shown in FIG. 3A. From the spectrum, ultrasound transducer 102 was most effective around 750 KHz, which was the design center frequency of this example aspect of ultrasound transducer 102. The −6-dB fractional bandwidth was estimated to be about 66% in the range from 420 to 835 kHz, where the lower frequency (i.e., 420 kHz) was chosen at the inflection point of the curve since the frequency response did not drop below-6 dB in the lower frequency region. Frequencies 450 kHz, 500 KHz, 550 kHz, 600 kHz, and 650 kHz were selected for testing, which represents relatively high sensitivities as the secondary frequency components combined with the center frequency (i.e., 750 kHz).

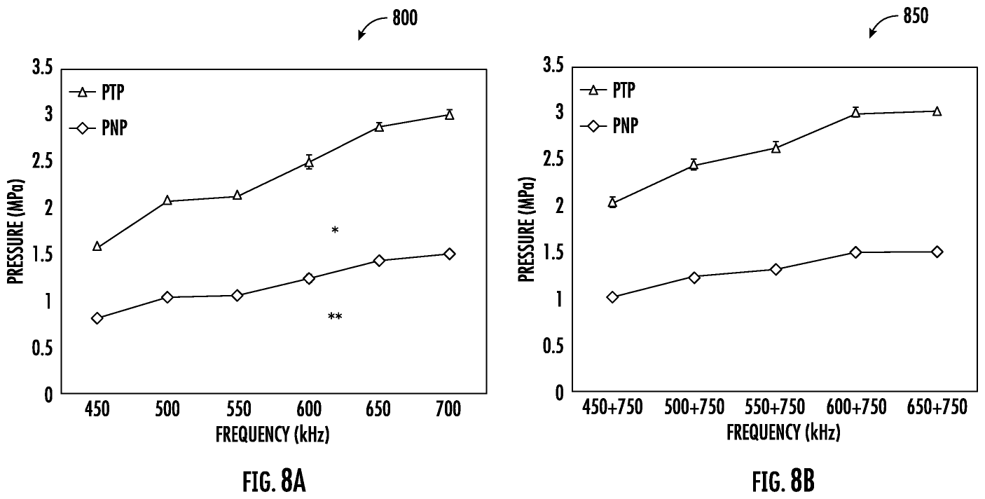

FIG. 8A shows a graph 800 of the measured PTP and PNP from the chosen single frequency of 750 kHz under an input voltage of 80 Vₚₚ. As shown in FIG. 8A, the PTP can reach up to 3.04 MPa at 750 kHz with a corresponding PNP of 1.51 MPa. The results also indicate the drop of the pressure output for the frequencies lower than 750 kHz. The 600- and 650-kHz results showed a pressure level above 2.5 MPa for the PTP and 1.3 MPa for the PNP: 500 and 550 kHz showed similar efficiency with a PTP over 2.2 MPa and PNP over 1.1 MPa. However, 450 kHz was less effective with a PTP of 1.6 MPa and a PNP of 0.8 MPa. Therefore, 450 kHz was chosen as the lower limit for the frequency combinations.

FIG. 8B shows a graph 850 of the measured pressure level from the various frequency combinations with a ratio of 1:1 to each frequency component; 650 KHz+750 kHz and 600 kHz+750 kHz showed comparable pressure levels with the 750-kHz single-frequency cases. The pressure level with the low-frequency combinations (450 kHz, 500 kHz, and 550 kHz) increased adding the 750-kHz components with all PNP larger than 1.1 MPa, which was believed to be effective for generating cavitation with MBs. Based on the pressure characterization results, input voltage was weighted to ensure that each frequency component contributes the same acoustic power during the treatment. With the dual-frequency methods, the PNP pressure was 1.4 times larger than the single-frequency methods under the same power input.

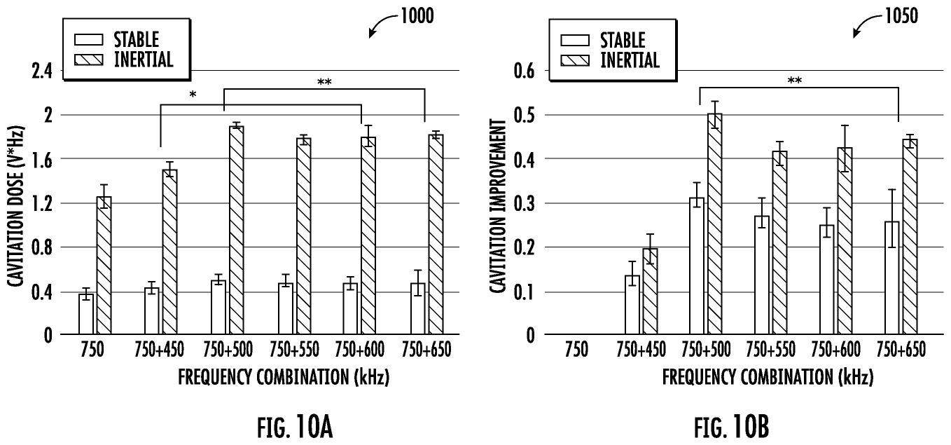

FIG. 9A shows a graph 900 of mass reduction for unretracted and retracted clots under frequency combinations. Notably for FIGS. 9A-10B, * signifies p<0.01 and ** signifies p<0.05. The clots underwent a 30-minute treatment of various frequency combinations using setup 400 shown in FIG. 4 for in vitro thrombolysis. The mass of the clots before and after the treatment was compared and the percentage of mass reduction in 30 min was defined as the thrombolysis rate. For the unretracted clots, the single-frequency method showed an averaged mass reduction of 43%, whereas the dual-frequency method showed an averaged mass reduction of 53%. FIG. 9B shows a graph 950 of dual-frequency treatment improvement (Id) compared with single-frequency treatment. FIG. 9B shows an averaged 46% improvement over the single-frequency treatment. For the retracted clots, similarly, the single-frequency treatment method showed an averaged mass reduction of 19% and the dual-frequency method showed an averaged mass reduction of 31%. The corresponding lysis rate improvement was estimated in FIG. 9B with the pattern-filled bar and the averaged improvement of 85% was obtained with the dual-frequency methods. Compared with unretracted clots, the dual-frequency methods showed more improvement on retracted clots sonothrombolysis.

FIG. 10A shows a graph 1000 of passive cavitation dose under various frequency combinations. The cavitation was monitored by passive cavitation detection and compared between the single-frequency and dual-frequency excitation methods. The increase of the cavitation dose was defined as (C_{d}−Cₛ)/Cₛ, where Cₛ represents the cavitation dose from the single-frequency test at 750 KHz and C_{d} represents the cavitation dose from the dual-frequency test with the chosen frequency combinations. Both stable and inertial cavitation doses were calculated, as shown in FIGS. 10A and 10B. FIG. 10A shows the cavitation doses from single-frequency and dual-frequency treatment cases.

FIG. 10B shows a graph 1050 of cavitation dose improvement with dual-frequency excitation. FIG. 10B illustrates the cavitation increase from the dual-frequency method compared with the single-frequency method. Compared with the single-frequency method, the dual-frequency method showed an average 24.9% increase for stable cavitation and a 40.1% increase for inertial cavitation. The results indicated similar cavitation doses for the 750 KHz+550 kHz, 750 KHz+600 kHz, and 760 KHz+650 kHz groups and a lower cavitation dose for 750 KHz+450 KHz and a relatively higher cavitation dose for the 750 KHz+500 kHz group.

The dual-frequency technique using ultrasound transducer 102 achieved effective treatment for both un-treatment and retracted clots with a lysis mass reduction up to 58% and 32%, respectively, after a 30 min treatment, corresponding to 46% and 85% lysis rate increase, respectively, compared with the single-frequency treatment method. The effect of unretracted and retracted clots in sonothrombolysis with the dual-frequency method was also demonstrated and compared. Thrombolysis rate with the dual-frequency excitation was more effective for retracted clots (85% improvement) than unretracted ones (46% improvement), which indicated that the dual-frequency treatment methods were more effective for retracted clots treatment. The improvement in the thrombolysis rate for retracted clots could be attributed to the increased cavitation dose.

The effect of different frequency combinations with larger frequency gaps in a range from 450 kHz to 750 KHz was also investigated. Among various frequency combinations, the 750 kHz+500 kHz showed the most significant increase (50.6% for retracted clots) in the thrombolysis rate. The result also supports the hypothesis that a large frequency difference potentiates the cavitation effect. For the combination of 750 kHz+550 kHz, 750 KHz+600 kHz, and 750 kHz+650 kHz, the thrombolysis rate and the cavitation dose showed similar results, which indicated that the frequency effect was not significant in this frequency range with a smaller frequency difference. Nonetheless, the 750 KHz+450 kHz condition did not show a high thrombolysis rate due to a relatively high electric impedance level of the transducer at 450 KHz. Although during the treatment, the waveform adjustment was added to make an equivalent comparison, the component voltage under 450 kHz excitation was not larger than 90 V considering the possibility of damaging the transducer, and therefore, the power output dropped with the 450 KHz frequency involved during the 30-min treatment. Comparing FIGS. 9A and 9B and FIGS. 10A and 10B, the lysis rate showed a consistent trend with the cavitation doses, which showed the highest lysis rate and highest cavitation doses under the 750 kHz+500 kHz combination. The cavitation showed the maximum value with the 750 KHz+500 kHz group, which also corresponds to the highest lysis rate in FIGS. 9A and 9B. With similar cavitation doses for 750 KHz+550 KHz, 750 KHz+600 kHz, and 750 kHz+650 kHz, the lysis rates were also at the same level for these three groups. With higher inertial cavitation doses and higher lysis improvement for the retracted clots, the results indicated a high potential for the deep vein sonothrombolysis applications where retracted clots are more common.

The described test results demonstrate the design, fabrication, and characterization of an aspect of ultrasound transducer 102 with a center frequency of 750 kHz and a footprint size of 1.4 mm. With a broad-6-dB bandwidth of 68.1%, the transducer was applied for the intravascular dual-frequency thrombolysis in a relatedly larger frequency range from 450 kHz to 750 kHz. The thrombolysis effect of the transducer was confirmed through an in vitro flow model providing a stable pressure, temperature, and flow rate environment. Compared with single-frequency treatment method, the dual-frequency treatment method increased the PNP and decreased the cavitation threshold under the same power, which leads to a higher cavitation efficiency and higher lysis rate. Besides, through various frequency study, a larger frequency difference between the two frequency components was expected to provide higher cavitation dose and higher lysis rate. By comparing the results from the unretracted and retracted clots, the dual-frequency method has shown a higher improvement for the sonothrombolysis. Since the dual-frequency method provides higher initial cavitation with MBs. this could be a potential method for breaking down retracted clots effectively. Moreover, the intravascular dual-frequency treatment required much lower power compared with other sonothrombolysis methods, which makes it promising for safe in vivo treatment.

These in vitro data support the effectiveness of the dual-frequency sonothrombolysis in safely improving the treatment of deep vein thrombosis. The intravascular device achieved high improvement in the treatment of both unretracted and retracted clots.

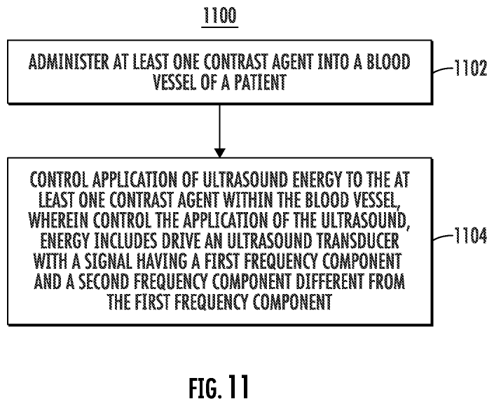

FIG. 11 is a flow diagram illustrating example method 1100 for sonothrombolysis mediated with contrast agents. At step 1102, at least one contrast agent is administered into a blood vessel of a patient.

At step 1104, application of ultrasound energy to the at least one contrast agent within the blood vessel is controlled, wherein controlling the application of the ultrasound energy includes driving an ultrasound transducer with a signal having a first frequency component and a second frequency component different from the first frequency component. Prior to application of the ultrasound energy, the ultrasound transducer may be inserted into the blood vessel adjacent to the at least one contrast agent. The ultrasound transducer may be housed in a catheter. The first frequency component may be a constant frequency component and the second frequency component may include a plurality of frequency steps within a range of frequencies. The first frequency component may have a center frequency of about 750 kHz and the frequency steps range from about 450 kHz to about 650 kHz at 50 kHz intervals. Contrast agents may include nanodroplets and/or microbubbles. A strength of the signal may be adjusted according to each frequency component. The strength of the signal may be adjusted to provide approximately equal levels of ultrasound energy by the first frequency component and the second frequency component of the signal. The ultrasound transducer may include at least one piezoelectric element. The ultrasound transducer may include a forward-viewing ultrasound transducer.

It will be understood that various details of the subject matter described herein may be change without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for sonothrombolysis mediated with contrast agents, the method comprising:
administering at least one contrast agent into a blood vessel of a patient; and
controlling application of ultrasound energy to the at least one contrast agent within the blood vessel, wherein controlling the application of the ultrasound energy includes driving an ultrasound transducer with a signal having a first frequency component and a second frequency component different from the first frequency component.

2. The method of claim 1 comprising inserting the ultrasound transducer into the blood vessel adjacent to the at least one contrast agent before controlling the application of the ultrasound energy.

3. The method of claim 2 wherein the ultrasound transducer is housed in a catheter.

4. The method of claim 1 wherein the first frequency component is a constant frequency component and the second frequency component includes a plurality of frequency steps within a range of frequencies.

5. The method of claim 4 wherein the first frequency component has a center frequency of about 750 kHz and the frequency steps range from about 450 kHz to about 650 kHz at 50 KHz intervals.

6. The method of claim 1 wherein the at least one contrast agent comprises at least one nanodroplet and/or at least one microbubble.

7. The method of claim 1 wherein a strength of the signal is adjusted according to each frequency component.

8. The method of claim 7 wherein the strength of the signal is adjusted to provide approximately equal levels of ultrasound energy by the first frequency component and the second frequency component of the signal.

9. The method of claim 1 wherein the ultrasound transducer comprises at least one piezoelectric element.

10. The device of claim 11 wherein the ultrasound transducer comprises a forward-viewing ultrasound transducer.

11. A system for sonothrombolysis mediated with contrast agents, the system comprising:
an ultrasound transducer; and
a function generator connected to the ultrasound transducer, the function generator configured for controlling application of ultrasound energy to at least one contrast agent within a blood vessel, wherein controlling the application of the ultrasound energy includes driving the ultrasound transducer with a signal having a first frequency component and a second frequency component different from the first frequency component.

12. The system of claim 11 wherein the ultrasound transducer is configured for being inserted into the blood vessel adjacent to the at least one contrast agent.

13. The system of claim 12 wherein the ultrasound transducer is housed in a catheter.

14. The system of claim 11 wherein the first frequency component is a constant frequency component and the second frequency component includes a plurality of frequency steps within a range of frequencies.

15. The system of claim 14 wherein the first frequency component has a center frequency of about 750 kHz and the frequency steps range from about 450 kHz to about 650 kHz at 50 kHz intervals.

16. The system of claim 11 wherein the at least one contrast agent comprises at least one nanodroplet and/or at least one microbubble.

17. The system of claim 11 wherein the function generator is configured for adjusting a strength of the signal according to each frequency component.

18. The system of claim 17 wherein the function generator is configured for adjusting the strength of the signal to provide approximately equal levels of ultrasound energy by the first frequency component and the second frequency component of the signal.

19. The system of claim 11 wherein the ultrasound transducer comprises at least one piezoelectric element.

20. The system of claim 11 wherein the ultrasound transducer comprises a forward-viewing ultrasound transducer.
